(19) United States
(12) Patent Application Publication
(10) Pub. No.: US 2026/0256981 A1
(43) Pub. Date: Sep. 3, 2026

(54) BONE REGENERATION MATERIAL COMPRISING BIODEGRADABLE FIBERS CONTAINING TRICALCIUM PHOSPHATE PARTICLES

(71) Applicant: ORTHOREBIRTH CO., LTD., Kanagawa (JP)

(72) Inventor: Toshihiro Kasuga, Aichi (JP)

(73) Assignee: ORTHOREBIRTH CO., LTD., Kanagawa (JP)

(21) Appl. No.: 19/573,656

(22) Filed: Mar. 20, 2026

Related U.S. Application Data
(63) Continuation-in-part of application No. PCT/JP2024/032052, filed on Sep. 6, 2024.
(60) Provisional application No. 63/539,398, filed on Sep. 20, 2023.

(51) Int. Cl. A61L 27/12; A61L 27/04; A61L 27/18; A61L 27/58

(52) U.S. Cl. CPC A61L 27/12; A61L 27/047; A61L 27/18; A61L 27/58; A61L 2430/02

(57) ABSTRACT

A bone regeneration material is provided that comprises biodegradable fibers containing both β-tricalcium phosphate (β-TCP) particles and modified calcium phosphate (m-CP) particles. The material has a cotton-like form in which the biodegradable fibers are entangled with one another. The biodegradable fibers contain 35-85 wt % tricalcium phosphate particles and 15-65 wt % poly(lactic-co-glycolic acid) (PLGA) resin as a balance. The tricalcium phosphate particles include β-phase tricalcium phosphate particles (β-TCP particles) having a particle diameter of 0.5-5 m, and modified calcium phosphate particles (m-CP particles) obtained by pulverizing the β-TCP particles to a particle diameter of 0.1-0.5 m by bead milling and converting them into an amorphous phase, wherein the β-TCP particles and the m-CP particles are present in a weight ratio of 95-50:5-50. The biodegradable fibers are spun from a spinning solution prepared using an anhydrous solvent, whereby the m-CP particles are dispersed so as to surround the β-TCP particles within the fibers without aggregation.

### TECHNICAL FIELD

The present invention relates to a bone regeneration material comprising biodegradable fibers containing tricalcium phosphate particles.

### BACKGROUND ART

β-tricalcium phosphate (β-TCP) is widely used as a bone-forming factor in bone regeneration materials. Existing bone regeneration materials using β-TCP particles include granular types (Osferion®) and types incorporating β-TCP particles into biodegradable fibers (ReBOSSIS®). In the latter, as the biodegradable resin degrades, the contained β-TCP particles come into contact with body fluids, dissolve, and release calcium ions/phosphate ions to promote bone formation.

When a bone regeneration material is implanted into the affected site, the β-TCP particles dissolve upon contact with body fluids, releasing calcium ions/phosphate ions. To achieve early bone formation, it is desirable that the release of calcium ions/phosphate ions from the β-TCP particles begins as soon as possible after implanting the bone regeneration material at the affected site. On the other hand, since bone formation takes time, it is desirable that after implanting the bone regeneration material at the affected site, the calcium ions/phosphate ions are supplied gradually and continuously in the necessary amounts in accordance with the progress of bone formation at the affected site.

Furthermore, as a means to promote bone formation at the affected site, it has been reported that supplying other biofunctional ions (such as silicon, boron, magnesium, etc.) in addition to calcium ions and phosphate ions further enhances bone formation at the site.

### PRIOR ART DOCUMENTS

### Patent Documents

Patent Document 1: U.S. Pat. No. 6,251,462, Patent Gazette

### Non-Patent Literature

Non-Patent Literature 1: Creation of a Metal Ion Controlled-Release Scaffold Material Based on the Activation Mechanism of Osteoblasts, Materia Vol. 59 No. 11 (2020), Akiko Obata, Toshihiro Kasuga

### SUMMARY OF THE INVENTION

### Problems to be Solved by the Invention

Under the above circumstances, there has been a need for a bone regeneration material that, after implantation into the affected area, would initiate the early release of calcium ions/phosphate ions and other ions effective for bone formation, and furthermore, would continuously release these ions during the progression of bone formation, thereby achieving effective bone formation. However, it was difficult to satisfy all these functions using crystalline β-TCP particles.

### Means for Solving the Problem

The inventor of the present invention, after diligent examination to solve the above problem, conceived of preparing particles (m-CP particles) by modifying the crystalline structure of β-TCP to amorphous using a bead mill to impact the β-TCP particles, and incorporating the m-CP particles together with β-TCP particles into a biodegradable fiber. Because β-TCP particles and m-CP particles have different dissolution rates, incorporating both enables early dissolution of calcium ions/phosphate ions from the m-CP particles, followed by continuous, sustained release of calcium ions/phosphate ions from the β-TCP particles. Furthermore, m-CP particles ionize upon impact, breaking crystal bonds and releasing negatively charged phosphate ions (PO₄³⁻). This allows coordination with positively charged metal or semimetal ions. However, when particle size becomes extremely small through bead milling, agglomeration within the fibers tends to occur. If particles agglomerate, it takes longer for the particles to come into contact with body fluids after implantation, making early bone formation difficult. The inventors of the present invention discovered that using an anhydrous solvent in preparing the spinning solution prevents particle agglomeration within the fibers and enables uniform dispersion.

Based on the above conception and discovery, the inventor of the present invention provides a bone regeneration material comprising biodegradable fibers containing tricalcium phosphate particles, wherein
the bone regeneration material comprises biodegradable fibers having an outer diameter of 60 to 200 m intertwined to form a cotton-like structure,
the biodegradable fibers contain 35 to 85 weight % tricalcium phosphate particles and 15 to 65 weight % polylactic-co-glycolic acid copolymer (PLGA resin) in the remainder,
the calcium phosphate contains, at a weight ratio of 95-50 to 5-50, β-phase tricalcium phosphate particles (β-TCP particles) having a diameter of 0.5-5 μm, and tricalcium phosphate particles (m-CP particles) that have been pulverized to a diameter of 0.1-0.5 μm by bead-mill processing of the β-TCP particles and modified into an amorphous phase, and
the biodegradable fiber is spun from a spinning solution prepared using an anhydrous solvent, wherein the m-CP particles are dispersed surrounding the β-TCP particles without agglomerating in the fiber.

Preferably, the biodegradable fibers contain tricalcium phosphate particles at 50 to 75 wt % and PLGA resin at 50 to 25 wt %, wherein the tricalcium phosphate particles contain the β-TCP particles and the m-CP particles in a weight ratio of 95-75:10-25.

Preferably, the PLGA resin is a PDLLGA resin containing D-isomers, and the anhydrous solvent is acetone.

Preferably, the PDLLGA resin is produced by co-polymerization of PDLLA and PGA in a weight ratio of 75-50:25-50. In this application, PDLLGA resin produced by co-polymerization of PDLLA and PGA in a weight ratio of 75:25 is denoted as PDLLGA (75:25), and PDLLGA resin produced by co-polymerization of PDLLA and PGA in a weight ratio of 50:50 is denoted as PDLLGA (50:50).

Preferably, the m-CP particles are prepared by wet grinding β-TCP particles using acetone.

Preferably, the m-CP particles contain metal ions (e.g., magnesium ions, silver ions) or semimetal ions (e.g., boron ions, silicon ions).

Preferably, the m-CP particles contain silver ions, and the bone regeneration material containing m-CP particles containing silver ions exhibits antibacterial properties.

Preferably, the biodegradable fibers of the bone regeneration material of the present invention are spun using a wet spinning method.

Preferably, the outer diameter of the biodegradable fibers of the bone regeneration material of the present invention is 80 to 160 μm, more preferably 100 to 120 μm.

The inventor of the present invention further provides a method for manufacturing a bone regeneration material comprising biodegradable fibers containing tricalcium phosphate particles, the method comprising:
preparing modified tricalcium phosphate particles (m-CP particles) by griding β-phase tricalcium phosphate particles to a diameter of 0.1 to 0.5 μm using a bead mill;
preparing a spinning solution by mixing and stirring 30-80 wt % of β-TCP particles with a diameter of 0.5-μm, 5-35 wt % of the m-CP particles, and 15-65 wt % of PLGA resin in acetone, spinning the spinning solution to produce biodegradable fibers using a wet spinning method, wherein the biodegradable fibers have an outer diameter of 60-200 μm, and the m-CP particles are dispersed around the β-TCP particles without agglomerating in the fiber, and
washing the spun biodegradable fibers with ethanol and drying the fibers at room temperature to recover a bone regeneration material that are formed by the biodegradable fibers entangling with each other to form a cotton-like structure.

Preferably, the biodegradable fibers contain 40-70 wt % of β-TCP particles, 5-15 wt % of m-CP particles, and 15-65 wt % of a PLGA resin.

Preferably, the PLGA resin is a PDLLGA resin containing D-isomer, and the anhydrous solvent is acetone.

Preferably, the m-CP particles are prepared by wet grinding β-TCP particles using acetone.

Preferably, the method comprises a step of incorporating metal ions or semimetal ions into the m-CP particles.

### Effect of the Invention

In the bone regeneration material produced by a method of one embodiment of the present invention, m-CP particles are dispersed around β-TCP particles within fibers without agglomeration of the small m-CP particles. After implantation of the bone regeneration material into the affected site, the m-CP particles dissolve upon contact with body fluids, rapidly releasing calcium ions/phosphate ions. Subsequently, the β-TCP particles dissolve, continuously and slowly releasing calcium ions/phosphate ions. This enables effective bone formation. phosphate ions, thereby enabling effective bone formation.

The bone regeneration material produced by the method of one embodiment of the present invention uses PDLLGA resin, which has a fast degradation rate, as the biodegradable fiber. Therefore, after the bone regeneration material is implanted into the affected area, the PDLLGA resin comes into contact with body fluids and degrades early, causing calcium ions/phosphate ions to be released early.

The biodegradable fibers constituting the bone regeneration material manufactured by the method of one embodiment of the present invention are spun using a spinning solution prepared by dissolving PDLLGA resin in anhydrous acetone. This prevents tricalcium phosphate particles from binding each other and avoids particle aggregation in the fibers.

The m-CP particles contained in the biodegradable fibers of the bone regeneration material of the present invention have a small particle size of 0.1 to 0.5 μm and a large specific surface area. Furthermore, they are amorphized and negatively charged, enabling coordination of metal ions or semimetal ions such as magnesium ions, boron ions, silver ions, and silicon ions. When a bone regeneration material, in which these metal ions or semimetal ions are supported on the fibers, is implanted into a living body, the biodegradable fibers dissolve upon contact with body fluids. This allows the metal ions and semimetal ions, along with calcium ions and phosphate ions, to be eluted from the m-CP particles contained within the fibers.

The biodegradable fibers constituting the bone regeneration material manufactured by a method of one embodiment of the present invention are spun using a spinning solution prepared by dissolving PDLLGA resin in anhydrous acetone. This provides higher safety compared to using chlorinated solvents such as chloroform.

The biodegradable fibers constituting the bone regeneration material produced by a method of one embodiment of the present invention are spun using a wet spinning method, resulting in lower manufacturing costs compared to using an electrospinning method.

### BRIEF DESCRIPTION OF THE DRAWINGS

FIG. 1 shows a cotton-like bone regeneration material composed of the biodegradable fibers of the present invention.

FIG. 2 shows a conceptual diagram of the composition of the biodegradable fibers of the present invention containing β-TCP particles and m-CP particles.

FIG. 3 shows a cross-section of the biodegradable fibers of the present invention containing β-TCP particles and m-CP particles.

FIG. 4 shows a scanning electron microscope (SEM) image of a cross-section cut by a cooling cross-section polisher (CCP; IB19520CCP, JEOL Ltd.) of the biodegradable fiber of the present invention.

FIG. 5A shows a bead mill apparatus used in the present invention.

FIG. 5B shows a bead mill apparatus used in the present invention.

FIG. 6 shows the result of animal test using the samples of the bone regeneration material of the present invention.

### EMBODIMENTS OF CARRYING OUT THE INVENTION

The preferred embodiments for implementing the present invention will now be described with reference to the drawings.

### Definitions

<m-CP Particles>

In the present invention, “m-CP particles” refer to tricalcium phosphate particles modified to an amorphous phase by grinding β-phase tricalcium phosphate particles to a diameter of 0.1 to 0.5 μm using a bead mill process. The m-CP particles enable negatively charged phosphate ions, released by the breaking of the calcium phosphate crystal structure, to coordinate with positive ions separated from metal or semimetal salts.

The tricalcium phosphate particles contained in the biodegradable fiber of the present invention contains β-TCP particles and m-CP particles in a weight ratio of 95-50 to 5-50. However, if the amount of m-CP particles relative to β-TCP particles is less than 5:95, the effect of high solubility of the m-CP particles is difficult to achieve. Conversely, if the ratio of m-CP particles to β-TCP particles exceeds 50:50, m-CP particle agglomeration intensifies. Furthermore, the ion elution rate from m-CP particles may temporarily become excessively high, potentially adversely affecting biological tissues.

### <Bead Mill Device>

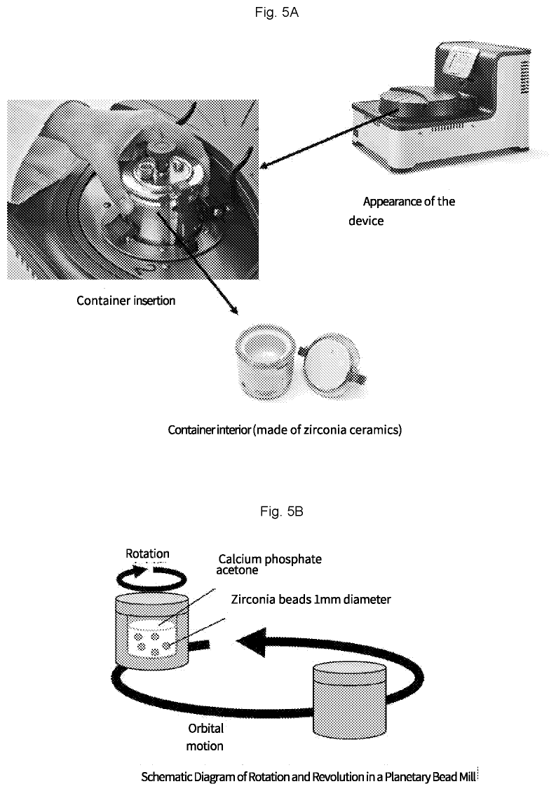

In the present invention, a “bead mill device” refers to a pulverizer used to apply mechanochemical processing to particles, employing beads with diameters of 0.015 to 3 mm as the grinding media (FIGS. 5A and 5B). In contrast to the 10 to 50 mm diameter balls used as grinding media in conventional ball mill devices, the beads used as grinding media in the bead mill device have a diameter of 0.015 to 3 mm. Using smaller diameter beads as grinding media enables imparting 100 to 500 times greater energy to calcium phosphate particles compared to using 10 to 50 mm diameter balls. Planetary bead mill devices achieve higher grinding efficiency by alternately rotating particles in a vessel through rotation and revolution, applying stronger impact energy to the particles via the grinding media beads. Although various types and models of bead mill devices exist, any of them can be used as the bead mill device of the present invention, provided they can finely grind calcium phosphate particles using beads.

### <Bead Mill Processing>

In the present invention, “bead mill processing” refers to the process of placing beads into the pot of a bead mill apparatus, introducing the powdered particles, and rotating it at high speed to impart significant energy to the particles constituting the powder through intense mechanical impact.

When significant energy is imparted to calcium phosphate particles through intense mechanical impact during bead milling, the particles are crushed, reducing their diameter, and the bonds within the particles break, causing ionization.

### <Wet Grinding>

In the present invention, “wet grinding” refers to a method of grinding by mixing a slurry, which is a mixture of powder and liquid, with beads in a container and agitating them. This is contrasted with dry grinding, which grinds without mixing liquid (in air). In bead mill processing, dry grinding causes progressively greater loading between the crushed particles, leading to clogging and the easy formation of hard agglomerates during grinding. Therefore, wet grinding is used in the bead mill processing of the present invention. In wet grinding, using a liquid that dissociates H⁺ and possesses polarity (e.g., water) causes the ground particles to hydrogen bond and agglomerate. Therefore, in order to avoid agglomeration of the ground particles, it is preferable to use a liquid that does not dissociate H and possesses no polarity (e.g., acetone).

### <Wet Spinning Method>

“Wet Spinning” generally refers to a method by which a polymer dissolved in a solvent is extruded through a nozzle in a liquid medium to form fibers. The wet spinning method used in the present invention is described in detail in WO2022/113888 (Method for Producing Cotton-Shaped Bone Regeneration Material) and WO2023/228905 (Method for Continuously Producing a Biodegradable Fiber Material Containing Inorganic Filler Particles Using a Wet Spinning Method, and Cotton-Shaped Bone Regeneration Material Produced by the Method).

### Embodiment 1

Manufacturing of a Bone Regeneration Material Comprising Biodegradable Fibers Containing β-TCP Particles and m-CP Particles

1. Preparation of m-CP Particles
Powdered β-TCP particles with a diameter of 0.5 to 5 μm are introduced into a zirconia pot (volume capacity 30 to 60 mL) of a planetary bead mill apparatus, together with 35 to 50 g of beads with a diameter of 0.5 to 1.4 mm, in an apparent volume ratio of 1:2 to 1:4. Add 15 to 20 mL of acetone and wet-grind by rotating the planetary bead mill at a predetermined speed.
Impact from the beads crushes the β-TCP particles, reducing their particle size to 0.1 to 1.2 μm and increasing their specific surface area by 3 to 5 times. Concurrently, the crystalline structure of the β-TCP particles partially amorphizes, reducing their crystallinity to 50 to 10%.
   2. Spinning of Biodegradable Fibers Containing β-TCP Particles and m-CP Particles

### 1) Preparation of Spinning Solution

Mix β-TCP particles, m-CP particles, and PDLLGA in a weight ratio of 30-55:5-35:15-65. Dissolve the polymer in acetone, mix overnight, and prepare a spinning solution with a polymer concentration of 17 to 23%. If the PDLLGA resin content is less than 15 wt %, the resin struggles to bind the particles as a binder, making spinning difficult. Conversely, if the PDLLGA resin content exceeds 65 wt %, the biocompatibility of calcium phosphate cannot be effectively utilized.

### 2) Wet Spinning

Spin the biodegradable fibers using the wet spinning method, employing the particle dispersion solution prepared above as the spinning solution.

### 3. Recovery of Cotton-Like Bone Regeneration Material

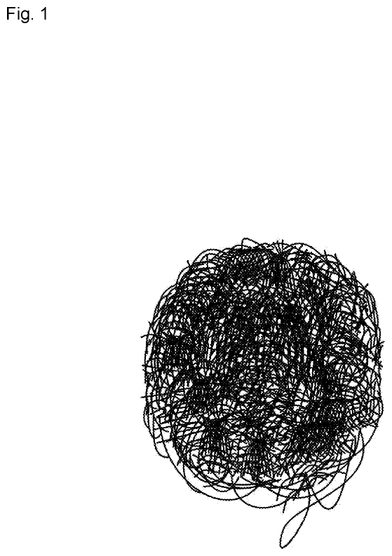

After wet spinning, the entangled biodegradable fibers are placed on an absorbent sheet to roughly remove the diluent (water, etc.). It is then dried overnight at room temperature to obtain cotton-like sample 1 (see FIG. 1).

### 4. Properties of the Obtained Biodegradable Fiber/Bone Regeneration Material

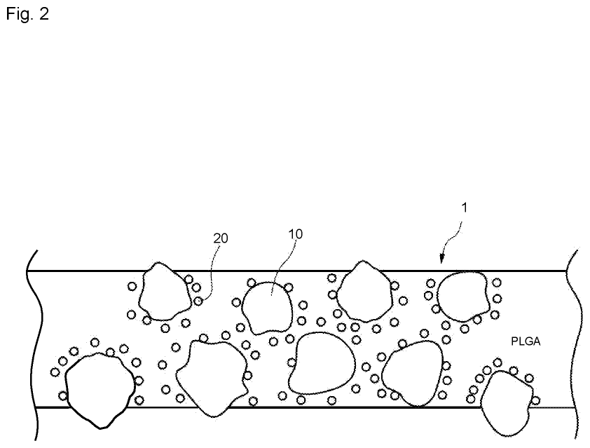

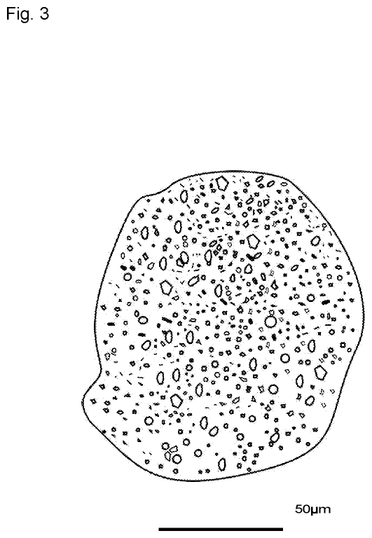

Biodegradable fibers with diameters of 60 to 200 μm are entangled to form a cotton-like shape.
Within the biodegradable fibers, the m-CP particles are dispersed around the β-TCP particles without agglomerating (see FIGS. 2 and 3).

### Embodiment 2

Manufacture of a bone regeneration material comprising biodegradable fibers containing β-TCP particles and m-CP particles containing metal or semimetal ions

1. Preparation of m-CP Particles Containing Metal or Semimetal Ions
(1) Mix β-TCP particle powder and metal (e.g., magnesium, silver) or semimetal (e.g., silicon, boron) salt particle powder in a weight ratio of 0.99-0.90:0.01-0.1. Place the powder mixture into the zirconia vessel of a planetary bead mill device (volume capacity 30 to 60 mL) together with 15-20 mL of acetone and 35-50 g of beads with a diameter of 0.6-1.4 mm. Rotate and oscillate the planetary bead mill device at a predetermined speed.
(2) Impact from the beads crushes the β-TCP particles, reducing their particle size and increasing their specific surface area. Simultaneously, the crystalline structure of the β-TCP particles partially amorphizes, transforming them into m-CP particles. Metal or semimetal salt particles introduced into the zirconia vessel of the planetary bead mill device are impacted by the beads, causing metal or semimetal ions to separate from the particles. The separated positive ions coordinate with the phosphate ions (negative charge) of the m-CP particles.
(3) After maintaining the planetary bead mill device at high rotation speed for a set period and then allowing it to stand, the m-CP particles are fixed in a state where ions separated from the metal or semimetal salt particles are coordinately bonded to the phosphate ions. Subsequently, separate the beads from the mixed powder by sieving and washing. The mixture is then placed in a 70° C. oven overnight to remove acetone, recovering m-CP particles containing metal or semimetal ions.
2. Spinning biodegradable fibers containing β-TCP particles and m-CP particles, recovery of cotton-like bone regeneration material, and properties of the obtained biodegradable fiber/bone regeneration material β-TCP particles, m-CP particles containing metal or semimetal ions, and PDLLGA were mixed in a weight ratio of 30-55:5-35:15-65, dissolving them in acetone to prepare a spinning solution. The spinning solution was spun into biodegradable fibers using a wet spinning method. The spun biodegradable fibers were dried to obtain a cotton-like sample 2 (see FIG. 1).

The properties of the obtained biodegradable fiber/bone regeneration material are the same as in Example 1, except that the m-CP particles contain metal or semimetal ions.

### Experiment 1

### <Outline of Experiment 1>

Preparation of biodegradable fibers containing β-TCP particles, m-CP particles, and PDLLGA. β-TCP particles (Tahei Chemical Industries, diameter 0.5-5 μm, average 1.2 μm), m-CP particles (diameter 0.02-1.0 μm, average 0.4 μm), and PDLLGA were mixed in a weight ratio of 30:14:56, dissolved in acetone, mixed overnight, and prepared a spinning solution with a polymer concentration of 23.3%. Using the prepared spinning solution, biodegradable fibers with a diameter of 100-120 μm were spun by the wet spinning method. The spun biodegradable fibers were cross-sectioned using a cooling cross-section polisher (CCP) (cooled to approximately −100° C. and ion milled by argon irradiation).

### <Result of Experiment 1>

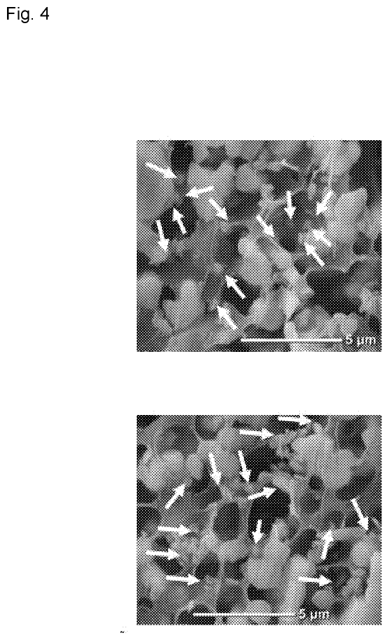

Images of the cross-section of the biodegradable fiber spun in the above experiment are shown in FIGS. 3 and 4.

In the SEM image of FIG. 4, the bright areas are tricalcium phosphate (TCP) particles, and the dark areas are PDLLGA resin. Since there is no chemical bond between the biodegradable fiber and the TCP, and their elastic moduli differ significantly, this grinding process subjected large TCP particles to stress, causing them to detach easily while leaving the matrix polymer behind, resulting in a cross-section riddled with holes.

Based on their size and shape, these holes are thought to be where TCP particles were originally embedded, with the polymer matrix portion remaining (the dark-appearing areas).

Since m-CP particles have a submicron-order particle size, those completely embedded within the matrix do not detach. The areas indicated by white arrows in FIG. 4 are thought to be m-CP particles located near the pore surfaces. This photograph shows m-CP particles dispersed around β-TCP particles.

### Experiment 2

### <Outline of Experiment 2>

Preparation of m-CP Particles Containing Silver Ions
(1) β-TCP powder and Ag₃PO₄ powder (both from Taihei Chemical Industries) were mixed at a weight ratio of 99:1 to prepare 1.8 g of mixed powder.
(2) Place 40.5 g of 1 mm diameter beads and 13.5 g (=17 ml) of acetone into a zirconia vessel (volume capacity 45 ml) of a planetary ball mill. With the beads/acetone added, the liquid level reached approximately 80% of the vessel's capacity. The prepared mixed powder (1.8 g) was added to the vessel. The planetary ball mill was then operated at high speed with an orbital speed of 400 rpm and a rotational speed of 800 rpm.
(3) High-speed rotation of the planetary ball mill finely pulverized the β-TCP particles. Concurrently, the impact from the beads partially amorphized the crystalline structure of the β-TCP particles. Silver ions separated from the Ag₃PO₄ particles due to the impact of the beads, and the separated silver ions (positively charged) coordinated with the phosphate ions (negatively charged) of the amorphized β-TCP particles (m-CP particles).
(4) After continuing the high-speed rotation of the planetary ball mill apparatus for 2 hours and then allowing it to stand, the m-CP particles were immobilized with silver ions separated from the Ag₃PO₄ particles coordinated around the phosphate ions.
(5) After the immobilization step, zirconia beads were separated from the powder mixture by sieving and washing, thereby recovering the m-CP particles containing silver ions.

### <Result of Experiment 2>

(i) Beads with a diameter of 1 mm were used. With 1 mm beads, they could be recovered with reasonable efficiency simply by washing with acetone after sieving through a sieve with an opening size of approximately 125 m. Using beads smaller than 1 mm increases the grinding effect but requires a sieve with a smaller opening size to prevent the beads from falling through, making recovery time-consuming.
(ii) After preparing particles by wet bead milling of Ag₃PO₄ particles and β-TCP particles, the resulting powder was immersed in a HEPES buffer solution. ICP-AES analysis of the solution showed that the silver ion concentration initially increased sharply before gradually decreasing.
(iii) The silver ion-containing m-CP particles prepared in Experiment 2 can be dispersed and incorporated into biodegradable fibers together with β-TCP particles, similar to Experiment 1.

### Experiment 3

### <Outline of Experiment 3>

Preparation of Boron Ion-Containing m-CP Particles
(1) A mixture of β-TCP powder (Tahei Chemical Industries) and B₂O₃ powder (Special Grade, Kishida Chemical) was prepared at a weight ratio of 10-30 to 90-70, yielding 1.8 g of mixed powder.
(2) Place 40.5 g of 1 mm diameter beads and 13.5 g (17 mL) of acetone into a zirconia vessel (45 mL volume capacity) of a planetary ball mill. With the beads/acetone inside, fill the vessel to approximately 80% capacity. The prepared mixed powder (1.8 g) was added to this mixture, and the ball milling apparatus was operated at high speed with an orbital speed of 400 rpm and a rotational speed of 800 rpm.
(3) High-speed rotation of the planetary ball mill partially amorphized the crystalline structure of the β-TCP particles due to impact from the beads. Simultaneously, the impact of the beads also caused strain, breakage, or new bonding in some of the B₂O₃ particle bonds, leading to their amorphization.
(4) After continuing the high-speed rotation of the planetary ball mill device for 2 hours and then allowing it to stand, the amorphized β-TCP (m-CP) was in a state where the amorphous B₂O₃ particles had fused with it.
(5) After the immobilization step, the treated material was separated by sieving and washing with acetone, recovering calcium phosphate particles containing boron ions.

### <Result of Experiment 3>

(i) The particle size of the particles obtained by co-grinding β-TCP powder and B₂O₃ powder was approximately 0.1 μm to 1 μm, with varied shapes. Furthermore, the number of agglomerated particles increased with increasing boron addition. I is considered that aggregation occurred due to boron reacting with atmospheric water during solid-liquid separation and drying, suggesting the powder reacted readily with moisture.
(ii) The m-CP particles containing boron ions prepared in Experiment 3 can be dispersed and incorporated into biodegradable fibers together with β-TCP particles.

### <Animal Experiment>

In order to evaluate in vivo performance of the bone regeneration material, animal experiment was conducted using rabbit posterolateral spine model.

Three types of cotton-wool like bone regeneration material samples (DL50-Ag, DL75-Ag, and DL50) manufactured by a wet spinning method were prepared.

In this experiment, DL50-Ag is an abbreviation for a bone regeneration material composed of biodegradable fibers containing m-CP particles carrying silver ions and β-TCP particles in PDLLGA (50:50), DL75-Ag is an abbreviation for a bone regeneration material composed of biodegradable fibers containing m-CP particles carrying silver ions and β-TCP particles in PDLLGA (75:25), and DL50 is an abbreviation for a bone regeneration material composed of biodegradable fibers containing β-TCP particles in PDLLGA (50:50) and not containing m-CP particles.

These three types of samples were implanted into the posterolateral spine of rabbits (7-month-old female New Zealand White rabbits), and fusion between bones was evaluated at 8 weeks and 12 weeks after implantation. To avoid errors due to individual differences among rabbits, each sample was implanted into the posterolateral spine of three rabbits, and the evaluation was comprehensively determined based on the spinal fusion observed in the three rabbits.

Composition of the three samples: DL50-Ag, DL75-Ag, DL50 is shown in Table 1.

TABLE 1

| | PDLLGA | β-TCP | m-CP:Ag |
|---|---|---|---|
| DL50-Ag | 30% | 63% | 7% |
| DL75-Ag | 30% | 56% | 14% |
| DL50 | 30% | 70% | 0% |

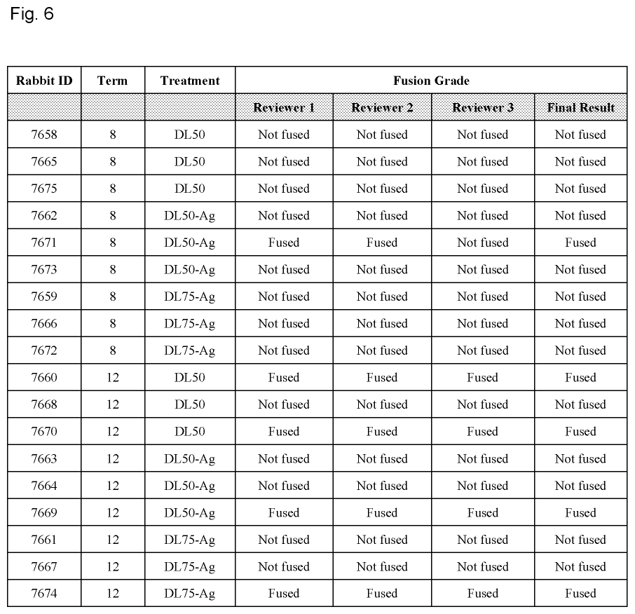

Radiographs were assessed for bilateral fusion at 8 and 12 weeks. Results are listed in FIG. 6.

Observations of test result are as follows:
(1) At 8 weeks, bone fusion was observed in one sample (ID No. 7671), and at 12 weeks, bone fusion was observed in four samples (ID Nos. 7669, 7674, 7660, and 7670). After implantation of the samples into the posterolateral spine, bone formation occurred at a relatively early stage, and as a result, bone fusion—i.e., union between adjacent bones—was observed at 8 and 12 weeks.
(2) At 8 weeks, bone fusion in the rabbit posterolateral spine was observed in one sample, and at 12 weeks, bone fusion was observed in four samples. This was a surprising result that such bone formation and bone fusion were achieved by implanting the bone regenerative material into the posterolateral spine without the use of autologous bone.
(3) No difference in bone fusion was observed between DL50-Ag and DL75-Ag, each carrying silver, and DL50 without silver. This suggests that loading silver onto the calcium phosphate particles did not inhibit bone formation.
(4) More bone fusion was observed in the DL50 series than in the DL75 series. Although the reason is not clear, it is presumed that the faster degradation of the PDLLGA resin increased the surface area of β-TCP particles exposed on the fiber surface, and that the DL50 series exhibited higher wettability with respect to water, thereby facilitating cell adhesion.

The present invention has been described with reference to specific examples, but it is not limited to these examples. The scope of the invention encompasses any structure or method described in the appended claims. Furthermore, the spinning method for the biodegradable fiber is not limited to the wet spinning method described in the examples. As long as it is possible to spin a biodegradable fiber containing both β-TCP particles and m-CP particles, the use of other spinning methods, such as electrospinning, also falls within the scope of the present invention.

### SYMBOL EXPLANATION

1 Biodegradable fiber
10 β-TCP particle
20 m-CP particle

1. A bone regeneration material comprising biodegradable fibers containing tricalcium phosphate particles,
wherein the bone regeneration material has a cotton-like form constituted by biodegradable fibers having an outer diameter of 60 to 200 μm and entangled with each other,
wherein the biodegradable fibers contain 35 to 85 wt % of tricalcium phosphate particles and 15 to 65 wt % of PLGA resin as a remainder,
wherein the tricalcium phosphate particles comprise: R-phase tricalcium phosphate particles (β-TCP particles) having a particle diameter of 0.5 to 5 μm, and modified tricalcium phosphate particles (m-CP particles) obtained by bead-milling the β-TCP particles to a particle diameter of 0.1 to 0.5 μm and converting the particles into an amorphous phase,
the β-TCP particles and the m-CP particles being present in a weight ratio of 95-50:5-50 in the biodegradable fibers, and
wherein the biodegradable fibers are spun from a spinning solution prepared using an anhydrous solvent, and the m-CP particles are dispersed so as to surround the β-TCP particles without aggregation within the fibers.

2. The bone regeneration material of claim 1,
wherein the biodegradable fibers contain 50 to 75 wt % of tricalcium phosphate particles and 25 to 50 wt % of PLGA resin,
and wherein the tricalcium phosphate particles comprise the β-TCP particles and the m-CP particles at a weight ratio of 90-75:10-25.

3. The bone regeneration material of claim 1,
wherein the PLGA resin is PDLLGA resin containing D-isomer, and the anhydrous solvent is acetone.

4. The bone regeneration material of claim 1,
wherein the m-CP particles are prepared by wet-milling the β-TCP particles using acetone.

5. The bone regeneration material of claim 1,
wherein the m-CP particles carry metal or metalloid ions.

6. The bone regeneration material of claim 5,
wherein the m-CP particles carry silver ions.

7. The bone regeneration material of claim 1,
wherein the biodegradable fibers are produced by a wet spinning method.

8. The bone regeneration material of claim 3,
wherein the PDLLGA resin is PDLLGA (50:50) resin.

9. A method for producing a bone regeneration material comprising biodegradable fibers containing tricalcium phosphate particles, the method comprising:
bead-milling β-phase tricalcium phosphate particles (β-TCP particles) to obtain modified tricalcium phosphate particles (m-CP particles) having a particle diameter of 0.1 to 0.5 μm and modified to an amorphous phase;
preparing a spinning solution by stirring and mixing 30 to 80 wt % of β-TCP particles having a particle diameter of 0.5 to 5 μm, 5 to 35 wt % of the m-CP particles, and 15 to 65 wt % of PLGA resin in acetone;
spinning biodegradable fibers from the spinning solution by a wet spinning method; and
washing the spun biodegradable fibers with ethanol and drying at room temperature to recover the bone regeneration material in a cotton-like form.

10. The method of claim 9,
wherein the m-CP particles are prepared by wet-milling the β-TCP particles using acetone.

11. The method of claim 9,
further comprising a step of loading metal or metalloid ions onto the modified tricalcium phosphate particles.
